(19) United States
(12) Patent Application Publication
(10) Pub. No.: US 2024/0085417 A1
(43) Pub. Date: Mar. 14, 2024

(54) PERIPHERAL BLOOD PHENOTYPE LINKED TO OUTCOMES AFTER IMMUNOTHERAPY TREATMENT

(71) Applicant: Memorial Sloan Kettering Cancer Center, New York, NY (US)

(72) Inventors: Margaret K. Callahan, Princeton, NJ (US); Arshi Arora, Brooklyn, NY (US); Taha Merghoub, Jersey City, NJ (US); Katherine S. Panageas, Ridgefield, CT (US); Michael A. Postow, Weehawken, NJ (US); Ronglai Shen, New York, NY (US); Jedd D. Wolchok, New York, NY (US)

(21) Appl. No.: 18/273,365

(22) PCT Filed: Jan. 20, 2022
(86) PCT No.: PCT/US2022/013109

§ 371 Date: Jul. 20, 2023

Related U.S. Application Data
(60) Provisional application No. 63/139,747, filed on Jan. 20, 2021.

(51) Int. Cl. G01N 33/569; A61P 35/00; C07K 16/28; G01N 15/14; G01N 33/574

(52) U.S. Cl. CPC G01N 33/56972; A61P 35/00; C07K 16/2803; C07K 16/2818; G01N 15/1429; G01N 33/57407; G01N 33/5743; A61K 2039/505; G01N 2333/70517

(57) ABSTRACT

Provided are methods of assigning a LAG+, LAG−, or PRO immunotype to a cancer patient based on the frequencies of LAG-3+CD8+T-cells, Ki67+CD8+T-cells, Tim-3+CD8+T-cells, and ICOS+CD8+T-cells in a peripheral blood sample from the patient, and selecting an anti-cancer therapy, for example, an immune checkpoint blockade (ICB) therapy, based on the patient's immunotype.

### CROSS-REFERENCE TO RELATED APPLICATIONS

This application claims benefit of U.S. Provisional Application No. 63/139,747, filed Jan. 20, 2021, the entirety of which is herein incorporated by reference.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### BACKGROUND

Antibodies that block immunologic checkpoints such as programmed cell death protein 1 (PD-1), its ligand (PD-L1), or cytotoxic T-lymphocyte-associated protein 4 (CTLA-4) target the immune system, enabling it to mount a successful anti-tumor response in a subset of patients. These antibodies can be used to treat a variety of cancers, including melanoma and urothelial carcinoma. For patients who are resistant to PD-1/L1 or CTLA-4 blockade, new agents targeting co-regulators of T-cell activation are in development.¹ Clinical trials of agents targeting lymphocyte-activation gene 3 (LAG-3), T-cell immunoglobulin domain and mucin domain 3 (TIM-3), and T-cell immunoreceptor with immunoglobulin and immunoreceptor tyrosine-based inhibition motif domains (TIGIT) have reported anti-tumor activity, although response rates appear modest, perhaps due to a lack of biomarkers for patient selection.²⁻⁷ Identifying patients unlikely to benefit from approved antibodies and suitable for new therapies or combinations is an important unmet need.

Molecularly targeted anti-cancer therapies have had notable success through the rational and precise selection of patients based on characterization of their tumors. For therapies that target the immune system, it has been challenging to determine which agents are most likely to benefit individual patients. The lack of biomarkers for immune checkpoint blocking (ICB) is a significant unmet need in oncology. Intratumoral expression of PD-L1 often correlates with response to PD-1/L1 blocking antibodies; however, PD-L1 has only been predictive in some studies and not others; its clinical use is therefore limited.^{8,9} Tumor mutation burden (TMB) has also been linked to outcomes in ICB treated patients with melanoma, UC, and other cancers but is not yet prospectively validated.⁹ A pre-treatment, blood-based biomarker that could guide clinical decision-making would be especially attractive.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

We conducted this study to profile the expression of targetable immune molecules expressed in the pre-treatment peripheral blood of cancer patients treated with ICB. Utilizing a large, clinically robust dataset with consistent banking and sample preparation methods, we applied innovative statistical and computational tools to multiparametric flow cytometry data to maximize the likelihood of signal detection. In this analysis, we identified and validated a pattern of pre-treatment peripheral blood markers that correlates with both response and survival in patients with melanoma and UC who received ICB.

Accordingly, the invention provides methods of assigning an immunotype of LAG+, LAG−, or PRO to a cancer patient based on the frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in a blood sample from the patient. The immunotype can then be used to guide treatment decisions, such that patients predicted to be susceptible or less susceptible to certain anti-cancer therapies are treated accordingly.

In one aspect, the invention provides a method of detecting a LAG+, LAG−, or PRO immunotype in a cancer patient, the method comprising: (a) conducting flow cytometry on a blood sample from the patient to determine normalized frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in the blood sample; and (b) implementing a classifier algorithm on a programmed computer, wherein the classifier algorithm uses a multinomial logistic regression to predict probabilities of the patient belonging to an immunotype of LAG+, LAG−, or PRO, by comparing the normalized frequencies determined in (a) with frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, from a training set of immunotype-labeled frequencies, wherein the training set is from a population of control cancer patients treated with immune checkpoint blockade (ICB) therapy; wherein the classifier algorithm assigns an immunotype of LAG+, LAG−, or PRO to the patient, based on the immunotype that has the highest predicted probability.

In another aspect, the invention provides methods of treating cancer in a patient, the method comprising: (a) prior to anti-cancer therapy, detecting an immunotype of LAG+, LAG−, or PRO in the patient by methods described herein; and (b) (i) if the patient has a LAG− immunotype, administering a PD-1 inhibitor monotherapy to the patient; or (ii) if the patient has a LAG+ or PRO immunotype, administering anti-cancer therapy that is not PD-1 inhibitor monotherapy.

The invention also provides methods for predicting the likelihood of a response to PD-1 inhibitor monotherapy in a cancer patient, the method comprising classifying the cancer patient as having an immunotype selected from LAG+, LAG−, and PRO, wherein the classifying comprises: (a) determining normalized frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in a blood sample from the patient; and (b) implementing a classifier algorithm that uses a multinomial logistic regression to predict probabilities of the patient belonging to an immunotype of LAG+, LAG−, or PRO, by comparing the normalized frequencies determined in (a) with frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, from a training set of immunotype-labeled frequencies, wherein the training set is from a population of control cancer patients treated with immune checkpoint blockade (ICB) therapy; wherein a cancer patient having a LAG− immunotype is predicted to be susceptible to PD-1 inhibitor monotherapy and wherein a cancer patient having a LAG3+ or PRO immunotype is predicted to be less susceptible to PD-1 inhibitor monotherapy.

In some embodiments of the invention, T cell frequency is measured using flow cytometry, for instance, fluorescence-activated cell sorting (FACS).

Some embodiments comprise treating a cancer patient having a LAG-immunotype with PD-1 inhibitor monotherapy. Some embodiments comprise treating a cancer patient having a LAG+ or PRO immunotype with anti-cancer therapy that is not PD-1 inhibitor monotherapy. Examples of anti-cancer therapy include, for example, any one or combination of surgery, radiation therapy, chemotherapy, immunotherapy, hormone therapy, CAR-T cell therapy, and stem cell therapy. In particular embodiments, a patient having a LAG+ or PRO immunotype is treated with immune checkpoint blockade (ICB) combination therapy or ICB monotherapy, wherein the ICB monotherapy is not PD-1 inhibitor monotherapy. In certain embodiments, a patient having a LAG+ immunotype is treated with a LAG-3 inhibitor, optionally in combination with one or more additional immune checkpoint inhibitors. In specific embodiments, a patient having a LAG+ immunotype is treated with anti-LAG-3/PD-1 combination therapy. Patients having a LAG+ or PRO immunotype can be treated with anti-CTLA-4 therapy, alone or in combination with another immune checkpoint inhibitor or with an anti-cancer therapy that is not an ICB therapy. For example, a patient having a LAG+ immunotype can be treated with anti-LAG-3/CTLA-4 combination therapy.

In some embodiments, the PD-1 inhibitor is selected from the group consisting of nivolumab, pembrolizumab, pidilizumab, and REGN2810. In some embodiments, the PD-1 inhibitor is a PD-L1 inhibitor selected from the group consisting of atezolizumab, avelumab, durvalumab, and BMS-936559. In some embodiments, the CTLA-4 inhibitor is selected from the group consisting of ipilimumab and tremelimumab. In some embodiments, the LAG-3 inhibitor is selected from the group consisting of EOC202, FS118, GSK2831781, INCAGNO2385, IMP321, LAG525, MGD013, MK-4280, REGN3767, relatilimab (BMS986016), Sym-022, and TSR-033.

In one embodiment, the cancer patient has melanoma. In another embodiment, the cancer patient has bladder cancer, such as urothelial carcinoma.

### BRIEF DESCRIPTION OF THE FIGURES

FIG. 1 shows that immunotype classification significantly correlates with survival in ICB-treated melanoma patients. Panel A shows survClust analysis of 78 flow cytometry parameters identified three peripheral blood immunotypes in the discovery dataset of melanoma patients treated with immune checkpoint blockade (ICB) (n=136). Panel B shows distribution of select individual flow cytometry parameters that characterize each immunotype. Panel C shows Kaplan-Meier analysis of overall survival by immunotype in the discovery dataset (n=136). Panel D shows Kaplan-Meier analysis of overall survival by immunotype in the subset of patients treated with anti-PD1 monotherapy in the discovery dataset (n=76). Panel E shows multivariate analysis of immunotype, stage, LDH, and liver metastases in the full cohort of melanoma patients (n=170; 170 patients in the 188-patient full dataset were evaluable for all parameters).

FIG. 2 shows selection of markers associated with the T-cell phenotype subclasses using penalized logistic regression. Panel A shows penalized logistic regression coefficient estimates for each marker as a function of the shrinkage parameter lambda discriminating the LAG+ from LAG− immunotype. Panel B shows penalized logistic regression coefficient estimates for each marker as a function of the shrinkage parameter lambda discriminating the LAG+ from PRO immunotype.

FIG. 3 shows representative examples of flow cytometry data for the 4 markers (LAG3+CD8+, TIM3+CD8+, Ki67+CD8+, ICOS+CD8+) included in the 4-marker classifier for melanoma patients (Panel A) and urothelial cancer (UC) patients (Panel B).

FIG. 4 shows survClust analysis of 4 flow cytometry parameters (LAG-3+CD8+T cells, Ki67+CD8+T cells, Tim-3+CD8+T cells, and ICOS+CD8+T cells) in 188 melanoma patients treated with ICB (Panel A); and Kaplan-Meier analysis of melanoma patients by immunotype (n=188) (Panel B).

FIG. 5 shows the relationship between immunotype and melanoma histology. Panel A shows Kaplan-Meier analysis of melanoma patients with cutaneous or unknown primary (excluding uveal, mucosal and acral histology) by immunotype (n=106). Panel B shows multivariate Cox regression evaluating the association of the immunotype with survival adjusting for histology and subsequent treatment in the melanoma discovery dataset (n=136).

FIG. 6 shows the predicted probability of belonging to each of the three immunotype subclasses for each urothelial cancer patient sample using a multinomial logistic regression on LAG3, KI67, TIM3, and ICOS on CD8 trained from the melanoma data set. The probability profile was sorted by LAG+ immunotype predicted probability from highest to lowest from left to right.

FIG. 7 shows that immunotype classification significantly correlates with survival and response in ICB-treated UC patients (n=94). Panel A shows a heatmap display of the 4-marker classifier (LAG-3+CD8+T cells, Ki67+CD8+T cells, Tim-3+CD8+T cells, and ICOS+CD8+T cells) in the validation cohort of UC patients treated with ICB. Panel B shows Kaplan-Meier analysis of overall survival by predicted immunotype in UC patients (n=94). Panel C shows Kaplan-Meier analysis of overall survival by predicted immunotype in the subset of UC patients treated with anti-PD1 monotherapy (n=67). Panel D shows multivariate analysis of immunotype, stage, LDH, and liver metastases in UC patients (n=93; 93 patients in the 94-patient full dataset were evaluable for all parameters).

FIG. 8 shows multivariate Cox regression evaluating the association of the immunotype clusters with survival adjusting for Belmont in UC patients who progressed after platinum-based chemotherapy (n=86).

FIG. 9 shows boxplots of tumor mutation burden and tumor expression of PD-L1 across the three immunotypes in melanoma patients (Panel A) or UC patients (Panel B).

FIG. 10 shows that LAG+ immunotype is associated with poorer outcomes in patients with favorable tumor markers, including PD-L1 and TMB. Panel A shows Kaplan-Meier analysis of overall survival in subset of PD-L1+ melanoma patients, stratified by LAG− immunotype (solid blue line) versus LAG+ immunotype (solid red line) compared to PD-L1-subset (dashed grey line). Panel B shows Kaplan-Meier analysis of overall survival in subset of PD-L1+ melanoma patients treated with anti-PD-1 monotherapy, stratified by LAG− immunotype (solid blue line) versus LAG+ immunotype (solid red line) compared to PD-L1-subset (dashed grey line). Multivariate analyses of immunotype, PD-L1, TMB, and ALC status for overall survival in all ICB-treated melanoma patients (Panel C), in PD-1 monotherapy treated melanoma patients (Panel D) or in ICB-treated UC patients (Panel E) are shown. Multivariate analyses of immunotype, PD-L1, TMB, and ALC status for response in all ICB-treated melanoma patients (Panel F), in PD-1 monotherapy treated melanoma patients (Panel G) or in ICB-treated UC patients (Panel H) are shown.

FIG. 11 shows flow cytometry gating strategy. Insets in pink are isotype controls.

### DETAILED DESCRIPTION OF THE INVENTION

Immune checkpoint blocking (ICB) antibodies are a cornerstone in cancer treatment. However, ICB benefits only a subset of patients. There are no validated blood-based biomarkers to refine predicted outcomes or guide treatment choices. We have identified blood-based correlates of clinical outcome that are relevant for ICB-eligible patients or ICB-resistant patients who may need alternative treatment.

As detailed below in the Examples, we performed immune profiling of 188 ICB-treated melanoma patients using multiparametric flow cytometry to characterize immune cells in patients' pre-treatment peripheral blood. A novel supervised statistical learning approach was applied to a discovery cohort to classify phenotypes and determine their association with survival and treatment response. An independent cohort of 94 ICB-treated urothelial carcinoma (UC) patients was used for validation.

We identified 3 distinct immune phenotypes (immunotypes), defined in part by the presence of a LAG-3+CD8+T-cell population. Melanoma patients with a LAG+ immunotype had poorer outcomes after ICB, with a median survival of 17.6 months compared to 75.8 months for those with the LAG− immunotype (P=0.04). The link between immunotype and clinical outcome was validated in UC patients; those with LAG+ versus LAG− immunotype differed in median survival (4.7 versus 27.5 months, P<0.001). Multivariate Cox regression and stratified analyses further show LAG+ immunotype is an independent marker of outcome when compared to known clinical prognostic markers and previously described biomarkers.

Prior to the present study, there were no validated biomarkers for ICB that make use of pre-treatment peripheral blood samples. Some tumor characteristics such as expression of PD-L1 and TMB have been correlated with clinical outcomes after ICB.¹⁵⁻²⁰ However, only PD-L1 has been validated as a companion diagnostic, and its clinical application is limited to selected scenarios.²¹⁻²⁵ Both TMB and PD-L1 testing require a tumor biopsy sample.

We show that for patients receiving PD-1/L1 blockade, unique information about their likelihood of response and survival can be detected from a peripheral blood sample collected pre-treatment. In particular, the pre-treatment peripheral blood LAG+ immunotype defines patients who are significantly less likely to benefit from ICB and provides a strategy for identifying actionable immune targets. The peripheral blood immunotype is independent of either TMB or PD-L1 status.

Stratifying patients by immunotype represents a step toward a more tailored, patient-specific approach to treatment with immunotherapy and testing new agents alone or in combination with existing PD-1/L1 and CTLA-4 inhibitors. The LAG-3+CD8+T-cells that help define the LAG+ immunotype suggest a rational mechanism underlying the poorer outcomes for this group and provide justification for clinically targeting LAG-3. LAG-3 is a unique checkpoint that is functionally distinct from and non-redundant with PD-1 and CTLA-4.^{26,27} Without wishing to be bound by theory, the anti-tumor response in patients with high levels of LAG-3-expressing peripheral T-cells may be inhibited in a way that PD-1 or CTLA-4 blockade cannot overcome.

LAG-3 has shown promise as a target in preclinical models; however agents targeting LAG-3 have shown only modest activity in unselected patient populations.^{2-4,6,28,29} This is a situation where biomarker selection could be especially impactful. For example, the anti-LAG-3 antibody relatlimab in combination with nivolumab had a response rate of 13% in an unselected population of melanoma patients, but the subset of patients whose tumors (presumably infiltrating lymphocytes) were positive for LAG-3 were most likely to benefit.⁴ The LAG+ immunotype described in this study can identify patients that are more likely to benefit from the combination of LAG-3 and PD-1 blockade.

Notable strengths of our study include a relatively large, clinically robust dataset; a reliable and uniform approach to PBMC banking and flow cytometry, coupled with bioinformatic adjustments for variability between batches of samples; and a machine learning algorithm for discovery. Furthermore, our analysis identifies a blood-based marker that correlates significantly with both survival and response outcomes in patients receiving ICB. None of the published studies examining PBMC samples of ICB treated cancer patients using single-cell analysis have identified the biomarker profiles identified herein.^{10,30-39}

This study offers evidence that characterization of a patient's peripheral blood prior to treatment initiation can provide actionable data to inform clinical decision-making, including the potential to identify immunotherapy targets relevant to individual cancer patients. Establishing a more precision medicine-like approach for patients considering ICB is clearly important as these medicines can have a salutary effect on survival, but may be accompanied by potentially fatal or morbid toxicities. The financial cost of cancer therapy also points to the need for a better means of patient selection. Our data provide patient-based evidence that LAG-3 plays a role in resistance to PD-1 blockade, supporting LAG-3 as a clinically relevant target, and identifying a subpopulation of cancer patients who can benefit most from LAG-3 blockade.

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values ±10% of the stated number. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

An “active agent” is an agent which itself has biological activity, or which is a precursor or prodrug that is converted in the body to an agent having biological activity. Active agents useful in the methods of the invention include, for example, inhibitors of immune checkpoint proteins or their ligand(s), including antibodies to immune checkpoint proteins or their ligands, which inhibit their function.

An “effective amount” of a composition as disclosed herein is an amount sufficient to carry out a specifically stated purpose. An “effective amount” can be determined empirically and in a routine manner, in relation to the stated purpose, route of administration, and dosage form.

The terms “inhibit,” “block,” and “suppress” are used interchangeably and refer to any statistically significant decrease in biological activity, including full blocking of the activity. An “inhibitor” is an active agent that inhibits, blocks, or suppresses biological activity in vitro or in vivo. Inhibitors include but are not limited to small molecule compounds; nucleic acids, such as siRNA and shRNA; polypeptides, such as antibodies, including monoclonal antibodies, or antigen-binding fragments thereof, dominant-negative polypeptides, and inhibitory peptides, such as peptide antagonists or agonists; and oligonucleotide or peptide aptamers.

The term “immune checkpoint blockade” or “ICB,” as used herein, refers to the administration of one or more inhibitors of one or more immune checkpoint proteins or their ligand(s). Immune checkpoint proteins include, but are not limited to, cytotoxic T lymphocyte-associated antigen 4 (CTLA-4), also known as CD152, programmed cell death protein 1 (PD-1), also known as CD279, lymphocyte-activation gene 3 (LAG-3), also known as CD223, T cell immunoglobulin mucin (TIM-3), also known as HAVcr2, and T cell immunoreceptor with Ig and ITIM domains (TIGIT).

A “CTLA-4 inhibitor” is an active agent that antagonizes the activity of CTLA-4 or reduces its production in a cell. Examples of CTLA-4 inhibitors include ipilimumab and tremelimumab, including derivatives thereof.

A “PD-1 inhibitor” is an active agent that antagonizes the activity of PD-1 or reduces its production in a cell. Examples of PD-1 inhibitors include cemiplimab, nivolumab, pembrolizumab, pidilizumab, REGN2810, and spartalizumab, and derivatives thereof. PD-1 inhibitors also include active agents that inhibit the PD-1 ligand (PD-L1), including atezolizumab, avelumab, durvalumab, and BMS-936559, including derivatives thereof.

A “LAG-3 inhibitor” is an active agent that antagonizes the activity of LAG-3 or reduces its production in a cell. Examples of LAG-3 inhibitors include EOC202, FS118, GSK2831781, INCAGNO2385, IMP321, LAG525, MGD013, MK-4280, REGN3767, relatilimab (BMS986016), Sym-022, and TSR-033, including derivatives thereof.^{47, 48}

A “TIGIT inhibitor” is an active agent that antagonizes the activity of TIGIT or reduces its production in a cell. Examples of TIGIT inhibitors include tiragolumab, including derivatives thereof.

ICB “monotherapy,” as used herein, is a treatment regimen in which one or more inhibitors of a single immune checkpoint protein are administered to a subject. For example, “PD-1 monotherapy” or “anti-PD-1 monotherapy” refers to treatment with only inhibitor(s) of PD-1, including inhibitors of PD-L1.

ICB “combination therapy” is a treatment regimen in which inhibitors of more than one immune checkpoint protein are administered to a subject. For example, “PD-1/CTLA-4 therapy,” “anti-PD-1/CTLA-4 therapy,” “PD-1/CTLA-4 combination therapy,” and so forth, refer to treatment with at least one inhibitor of PD-1 and at least one inhibitor of CTLA-4. Likewise, “LAG-3/PD-1 therapy,” “anti-LAG-3/PD-1 therapy,” “LAG-3/PD-1 combination therapy,” and so forth, refer to treatment with at least one inhibitor of LAG-3 and at least one inhibitor of PD-1.

As used herein, the term “gene expression signature” is used consistently with its conventional meaning in the art, and refers to an expression profile of a group of genes that is characteristic of a certain cell type, a certain cell population, a certain biological phenotype, or a certain medical condition. Gene expression signatures can be determined using any suitable method known in the art for determining the expression of a gene, including, but not limited to, those that detect and/or measure gene expression at the mRNA level or the protein level, such as RT-PCR-based methods, immunohistochemistry (IHC)-based methods, flow cytometry-based methods, and the like.

By “subject” or “individual” or “patient” is meant any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

A “control” patient or population is one that has not been subjected to methods of the invention. Control patients, or subjects in a control population, have the same disease or disorder as the patient being compared to the control population. For example, a clinical outcome of a cancer patient who is subjected to a method of the invention, e.g., a course of anti-cancer treatment based on assignment of immunotype, is compared with the average (median) outcome of subjects having the same type of cancer who were not subjected to methods of the invention, e.g., whose immunotypes were not considered in selecting an anti-cancer therapy.

Patients to whom the methods and uses of the invention can be applied may be undergoing anti-cancer therapy for any type of cancer. Examples include melanoma, skin carcinoma, non-small cell lung cancer (NSCLC), small cell lung cancer (SCLC), Merkle cell carcinoma (MCC), bladder cancer, kidney cancer, head and neck cancers, lymphoma, breast cancer, ovarian cancer, prostate cancer, pancreatic cancer, colorectal cancer, gastric cancer, and esophageal cancer. In one embodiment, the cancer is melanoma. In one embodiment, the cancer is bladder cancer, such as urothelial carcinoma. In one embodiment, the cancer is NSCLC. In one embodiment, the cancer is SCLC. In one embodiment, the cancer is pancreatic cancer. In one embodiment, the cancer is MCC.

Anti-cancer therapies include, for example, surgery, such as tumor resection surgery, radiation therapy, chemotherapy, immunotherapy, hormone therapy, CAR-T cell therapy, and stem cell therapy. In certain embodiments, the immunotherapy is ICB therapy, such as ICB monotherapy or ICB combination therapy.

Some embodiments of the present invention involve predicting a patient's response to ICB therapy based on the patient's immunotype and/or tailoring the patient's treatment regimen to the assigned immunotype. A patient's immunotype of LAG+, LAG−, or PRO can be assigned by determining the frequency of certain CD8+T-cell populations in a peripheral blood sample from the patient, wherein the CD8+T-cell populations consist of LAG-3+CD8+T-cells, Ki67+CD8+T-cells, Tim-3+CD8+T-cells, and ICOS+CD8+T-cells. Frequency of these four populations alone, without the need to consider other markers, can be used in determining a patient's immunotype of LAG+, LAG−, or PRO.

Cell frequency is measured as a percentage of a population of cells. For example, the frequency of LAG-3+CD8+T-cells in a sample is measured as a percentage of total CD8+T cells in the sample. Likewise, the frequencies of Ki67+CD8+T-cells, Tim-3+CD8+T-cells, and ICOS+CD8+T-cells in a sample are each measured as a percentage of total CD8+T cells in the sample. Cell frequency can be measured or quantified by any method known in the art. Examples of suitable techniques include, but are not limited to, those that involve immunohistochemistry (IHC), flow cytometry, and/or PCR, each of which technique can be used to detect, measure, and/or quantify cells having a given gene expression signature. In one embodiment, methods of the invention employ flow cytometry, including fluorescence-activated cell sorting (FACS), to measure T cell frequencies, such as CD8+T cell populations and sub-populations.

One of three immunotypes identified by the inventors can be assigned to a cancer patient based on the normalized frequencies of CD8+T-cells expressing one of four biomarkers. Each immunotype is qualitatively characterized by the frequency combination shown in Table I.

TABLE I
Peripheral Blood Immunotype

| | LAG+ | LAG− | PRO |
|---|---|---|---|
| LAG-3+CD8+ | high | low | high |
| Ki67+CD8+ | low | low | high |
| Tim-3+CD8+ | low | low | high |
| ICOS+CD8+ | low | low | high |

Immunotype is assigned using predicted probabilities from a multinomial regression. In particular, the immuno-phenotype classifier uses a weighted linear combination of normalized cell frequencies of the four markers shown in Table I to predict the probability that a patient sample belongs to a specific immuno-phenotype in a multinomial logistic regression framework.

A “training set” is an initial set of data used to fit the parameters of a classifier, i.e., to teach an algorithm how to process information to create a model. In methods of the present invention, the training set can be from a population of control patients.

The classifier used in the Examples is trained on a discovery set of 136 melanoma patient samples; each patient is assigned to the immuno-phenotype with the highest predicted probability. As an illustrative example, a patient has cell frequencies of 12.8% for LAG3+CD8+, 2.8% for Ki67+CD8+, 1.0% for Tim-3+CD8+, and 1.0% for ICOS+CD8+. After proper normalization of the cell frequencies, the predicted probabilities obtained from the multinomial logistic regression model for LAG+, LAG−, and PRO are 80%, 19%, and <1%, respectively. Therefore, the patient is assigned to the LAG+ immunotype.

The classifier algorithm is preferably implemented on a computer programmed to perform a multinomial logistic regression. The classifier algorithm is preferably a survClust algorithm.

Patients having the LAG+ or PRO immunotypes are less responsive to anti-PD-1 monotherapy, and can preferably be treated with another type of ICB therapy, for example, anti-LAG-3 monotherapy, anti-LAG-3/PD-1 combination therapy, or anti-CTLA-4/PD-1 combination therapy, or can be treated with another type of anti-cancer therapy that is not ICB therapy. Patients having the LAG− immunotype are more responsive to anti-PD-1 monotherapy, and can preferably be treated with PD-1 inhibitor monotherapy. The ability to assign an immunotype of LAG+, LAG−, or PRO by measuring the normalized frequencies of only four T-cell sub-populations in peripheral blood, and selecting a treatment based on the patient's assigned immunotype, represents an advance over prior methods.

T-cell frequency can be measured according to the methods of the invention at least about one, two, three, four, five, or six days or one, two, three, four, five, or six weeks prior to an anti-cancer therapy.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder if the patient shows, e.g., total, partial, or transient alleviation or elimination of symptoms associated with the disease or disorder.

A patient who is successfully treated with a therapy is “susceptible” to the therapy, whereas a patient who is not successfully treated with a therapy is “less susceptible” to the therapy.

In certain embodiments, a cancer patient subjected to a method of the invention is successfully treated if the patient's survival is longer than the median survival of patients having the same type of cancer as the cancer patient. For example, treatment of melanoma would be successful in a subject treated by the methods of the invention, i.e., wherein treatment selection was based on immunotype, if the subject survives longer than the median survival of melanoma patients who have not been treated by the methods of the invention, i.e., wherein treatment selection was not based on immunotype. Survival can be overall survival, i.e., length of time a patient lives, or progression-free survival, i.e., length of time a patient is treated without progression of the disease. Survival can be measured from the date of diagnosis or from the date that treatment commences. Overall survival, median overall survival, progression-free survival, and median progression-free survival can be determined by methods known in the art and/or by those described herein.

In certain embodiments a cancer patient subjected to a method of the invention is successfully treated if the patient has an improved response to the anti-cancer therapy compared with a patient having the same type of cancer who has not been subjected to a method of the invention. For example, treatment of bladder cancer would be successful in a subject treated by the methods of the invention, i.e., wherein treatment selection was based on immunotype, if the subject has an improved response compared to the median response of patients who have not been treated by the methods of the invention, i.e., wherein treatment selection was not based on immunotype. Response to anti-cancer treatment can be measured by known methods appropriate to the cancer type, for instance, using Response Evaluation Criteria in Solid Tumors (RECIST).⁴⁶ Patients evaluated using RECIST can have a complete response (CR), a partial response (PR), stable disease (SD), or progressive disease (PD). An improved response can also be assessed by other criteria, for example, duration of response, reduction in tumor volume, minimum residual disease (MRD), and the like.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1. Stratification of Patients by Immunotype

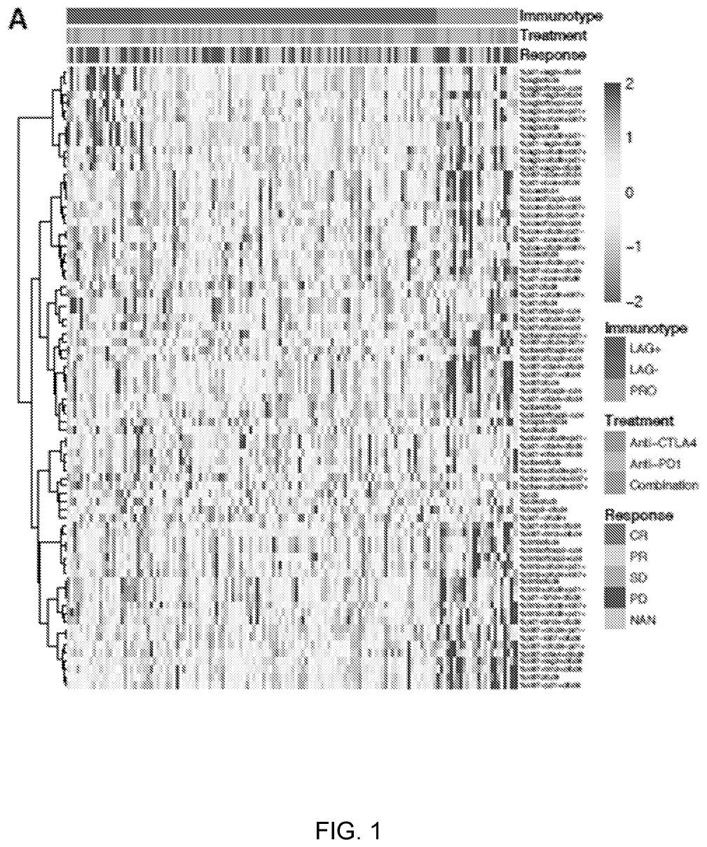

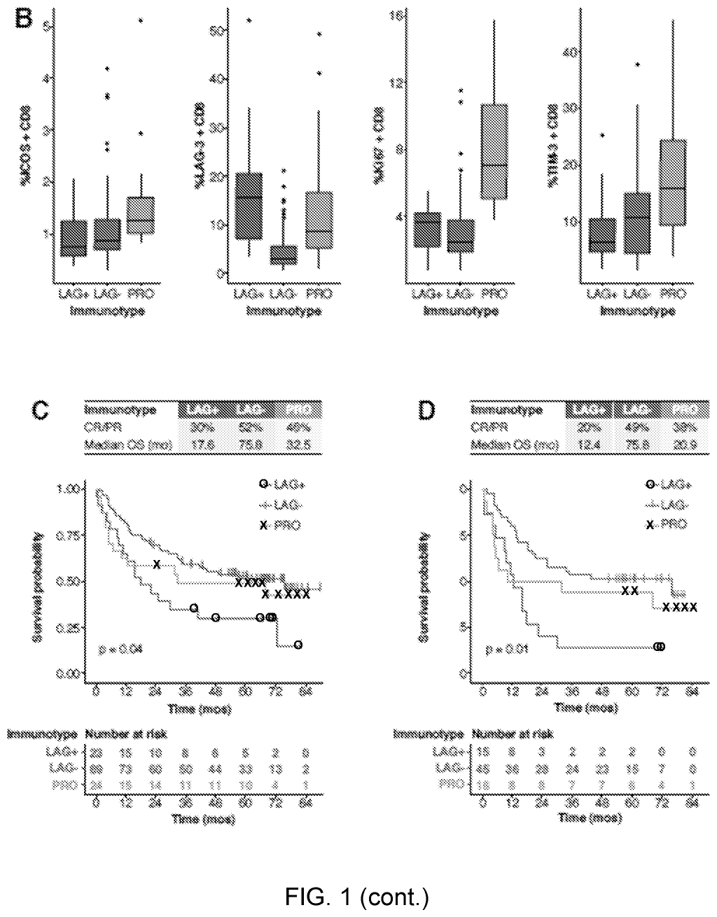

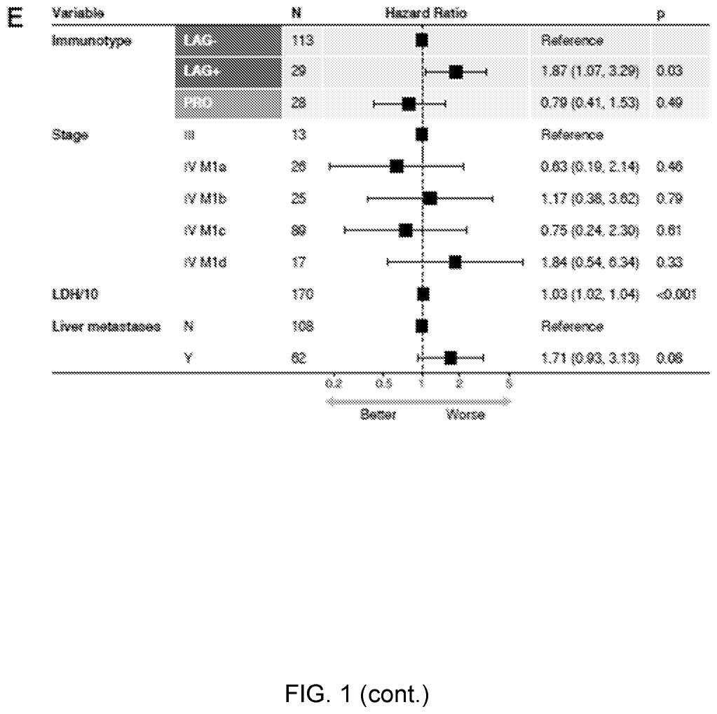

Our discovery cohort consisted of 136 ICB-treated melanoma patients. The median duration of follow up was 5.6 years. The clinical characteristics of this population are shown in Table 1 (Example 7); patients received either anti-PD-1 antibodies (n=76), anti-CTLA-4 antibodies (n=13), or both (n=47). We applied a supervised clustering analysis to stratify patients based on the multivariate pattern of 78 flow cytometry parameters from pre-treatment peripheral blood samples (FIG. 1, panel A). A rigorous cross-validation analysis revealed 3 clusters of patients with distinct patterns of expression of immune markers which we called ‘immunotypes.’

The first immunotype (LAG+) was uniquely characterized by high expression of LAG-3 on multiple T-cell populations, most representative of which was LAG-3+CD8+T-cells (FIG. 1, panel B). The LAG+ immunotype represented 17.0% (23/136) of patients. The second immunotype (LAG−) reflected 65.4% (89/136) of the population and was defined by a paucity of LAG-3+ cells and low levels of other co-markers on T-cells. The third immunotype had a high proportion of LAG-3+T-cells with concurrently high numbers of proliferating Ki67+CD8+T-cells and T-cells expressing TIM-3 and ICOS. We named this the proliferative (PRO) immunotype and it comprised 17.6% (24/136) of patients.

### Example 2. A Four-Marker Classifier to Define Immunotype

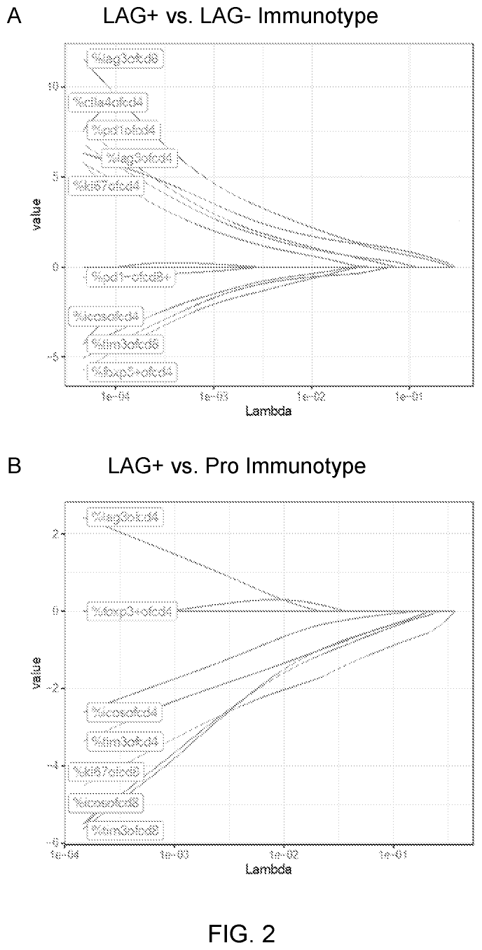

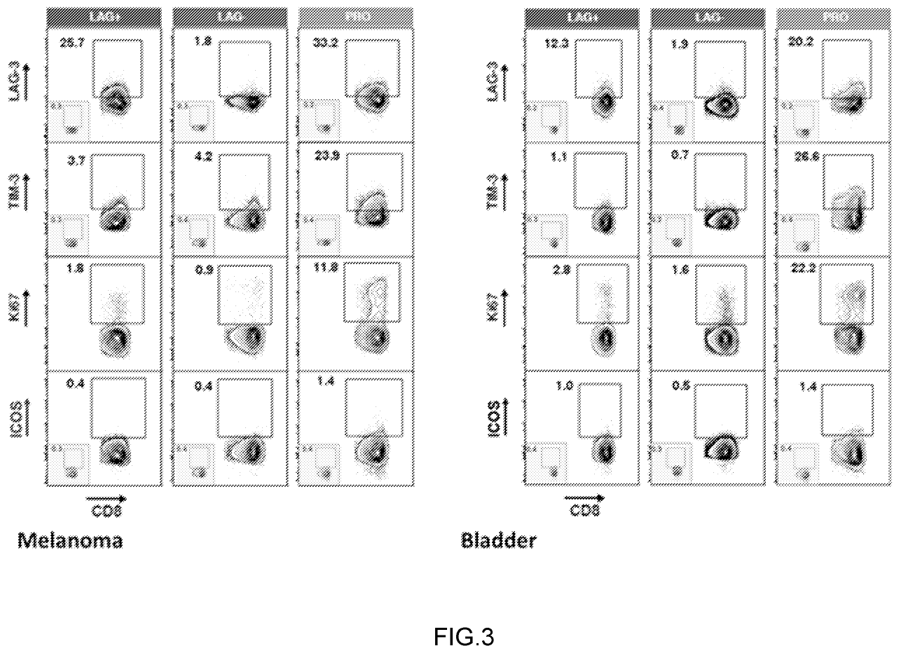

The initial clustering analysis used an input of 78 individual flow cytometry parameters to define three immunotypes; we next assessed the data to determine if a smaller number of parameters would be sufficient for this classification. First, we examined the flow markers that most significantly contributed to the classification of patients into specific immunotypes (FIG. 2). To identify the optimal combination of markers for classifying a sample into one of the immunotypes, we applied a penalized multinomial logistic regression approach¹³ and identified 4 markers (% LAG-3+CD8+T-cells, % Ki67+CD8+T-cells, % Tim-3+CD8+T-cells, and % ICOS+CD8+T-cells) that most strongly influenced the classification (FIG. 1, panel B; FIG. 3). Within this dataset, these 4 markers were able to reproduce the clustering assignment derived from the full panel of flow markers with 89% accuracy.

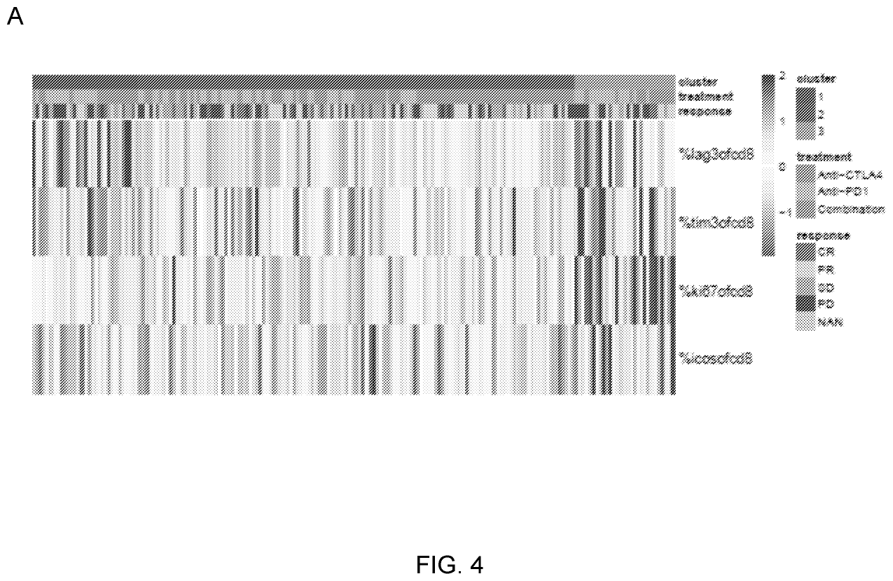

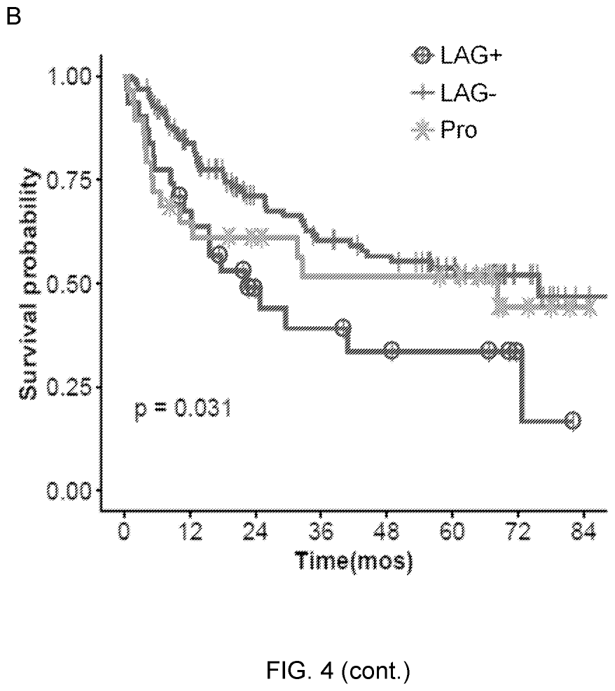

A multinomial logistic regression was then built, based on the selected 4 markers in our discovery dataset, to be taken forward for assigning an immunotype for future samples. This 4-marker classifier was applied to an additional 52 ICB-treated melanoma patients not available during the initial analysis of the discovery melanoma cohort. In the full melanoma dataset (n=188), the 3 immunotypes were distributed with 16.5%, 68.1%, and 15.4% patients classified within the LAG+, LAG−, and proliferative (PRO) immunotypes, respectively (FIG. 4; Table 2).

### Example 3. Immunotype Relates to Clinical Outcome in ICB-Treated Melanoma Patients

We next sought to determine if immunotype was related to response or overall survival after ICB treatment in the discovery dataset (FIG. 1, panel C). Melanoma patients with the LAG+ immunotype had the poorest outcome with median survival of 17.6 months compared to 75.8 and 32.5 months for those with either the LAG− or the PRO immunotype, respectively (P=0.04). Likewise, a clear trend of lower response rate in LAG+ patients was observed compared to the LAG− or PRO groups (30.4% versus 51.7% or 45.8%), although this did not reach statistical significance (P=0.18). This association between immunotype and survival outcome remained strong in the subset of patients who received anti-PD-1 monotherapy (n=76) with a median survival of 12.3 months for the LAG+ immunotype versus 75.8 and 20.9 months (P=0.01) in the LAG− and PRO immunotypes (FIG. 1, panel D). A significant relationship between immunotype and survival outcome was also evident in the full melanoma dataset including the 136 patients in the discovery dataset plus an additional 52 patients (n=188, P=0.03) (FIG. 4).

### Example 4. LAG+ Immunotype is Independent of Known Clinical Prognostic Factors

Table 1 presents the distribution of clinical characteristics of melanoma patients in the discovery dataset stratified by immunotype (for full melanoma dataset, see Table 2). The age, gender, and prior treatments were all well balanced. Melanoma substage, histology, liver metastases and levels of lactate dehydrogenase (LDH) were also evaluated. Of these factors, only LDH was significantly associated with immunotype (P=0.002).

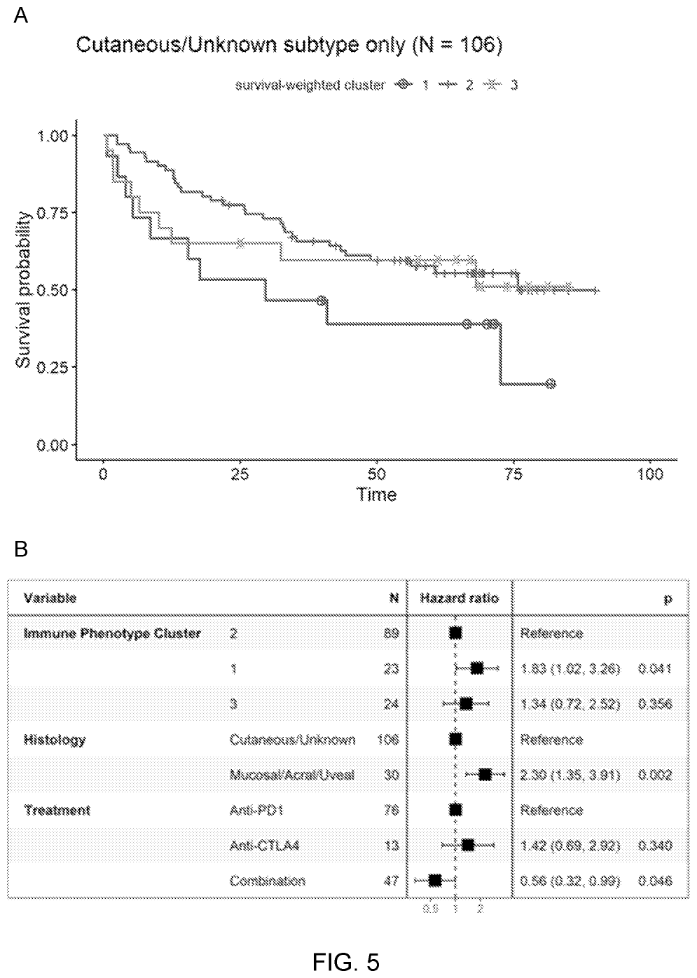

In multivariate analysis, the PRO immunotype no longer retained a significant association with survival after adjusting for LDH, liver metastases, and stage (FIG. 1, panel E). In contrast the LAG+ immunotype remained significant for its association with poor survival when analyzed in multivariate analyses adjusting for LDH, liver metastases, and stage (P=0.03) (FIG. 1, panel E) and in multivariate analyses adjusting for histology and subsequence treatment (P=0.04) (FIG. 5).

### Example 5. Immunotype Relates to Clinical Outcome in ICB-Treated Urothelial Cancer Patients

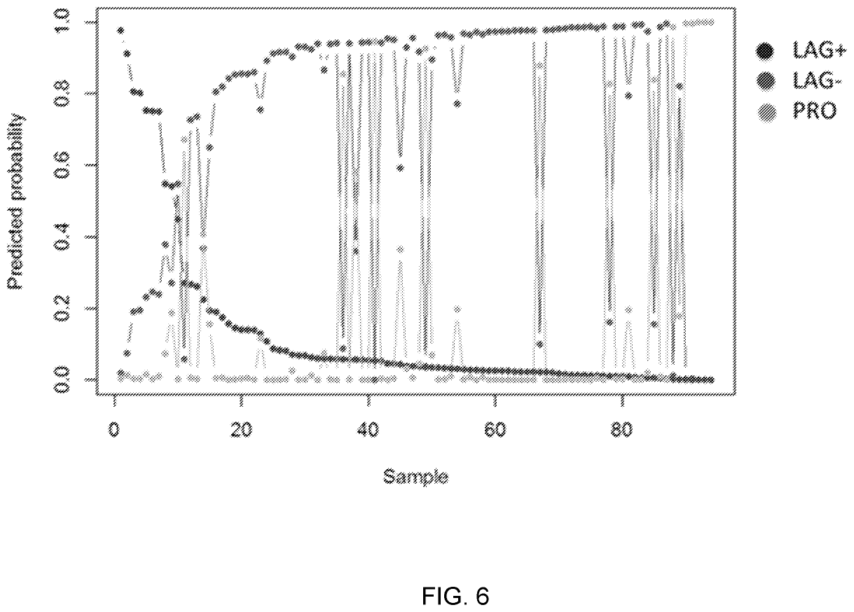

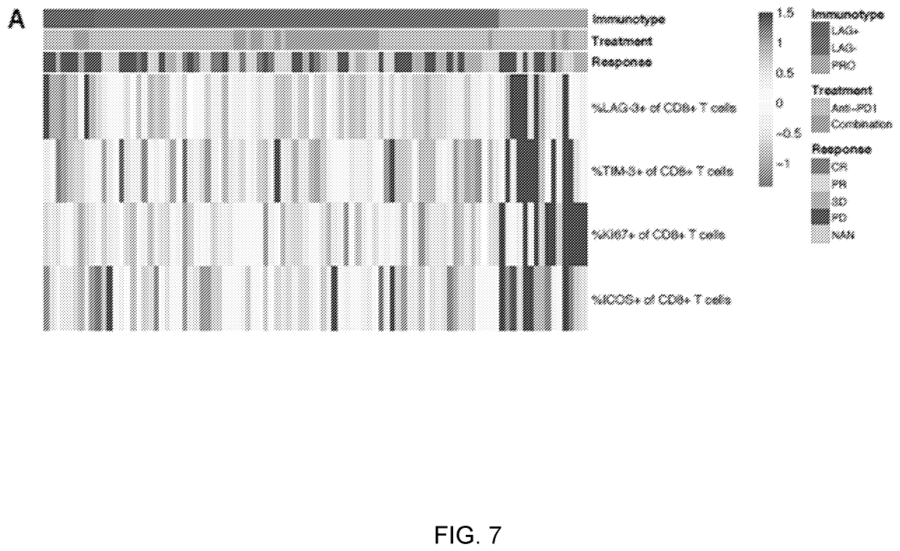

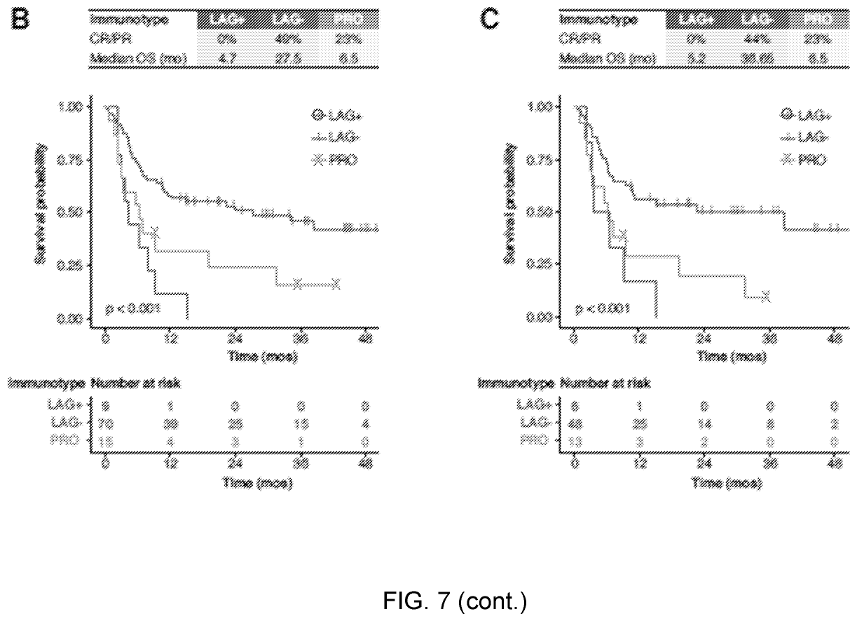

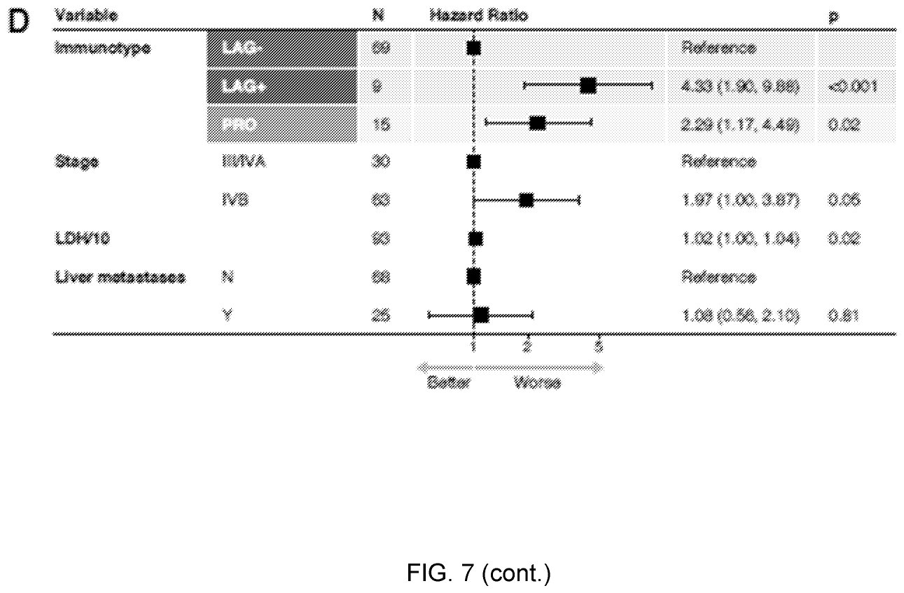

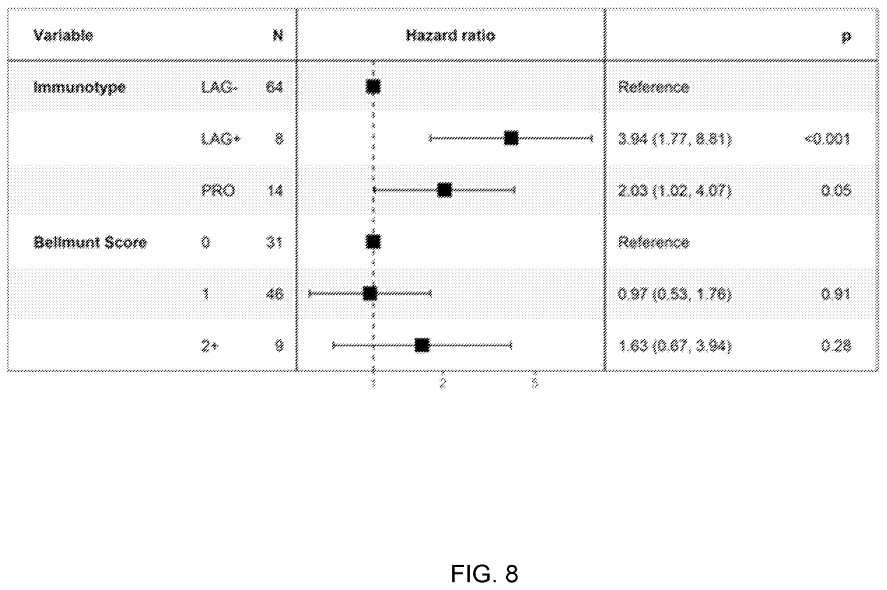

We applied the 4-marker classifier to flow cytometry data from pre-treatment peripheral blood samples in an independent cohort of 94 ICB-treated UC patients. Clinical characteristics of these groups are presented in Table 1. We assigned each UC patient to one of the immunotypes based on maximum predicted probability (FIG. 6). Clustering using this 4-marker classifier identified a similar distribution of the three immunotypes discovered in the melanoma cohort (FIG. 7, panel A). First, a subpopulation of patients (9.6%, 9/94) was defined by a higher proportion of LAG3+CD8+T-cells: the LAG+ immunotype. Second, a cluster with low levels of CD8+T-cells expressing LAG-3, Ki67, Tim-3 or ICOS, the LAG-phenotype, accounted for 74.5% (70/94) of the population. Finally, 16.0% (15/94) of patients had a high proportion of T-cells positive for Ki67, LAG-3, Tim-3, and ICOS: the proliferative (PRO) immunotype.

We then analyzed the clinical outcomes for UC patients in each of the immunotypes. Validating the observation in melanoma patients, UC patients with the LAG+ immunotype have particularly poor outcomes with a median OS of 4.7 compared to 27.7 and 6.5 months for the LAG− and PRO immunotypes, respectively (P<0.001) (FIG. 7, panel B). Patients categorized in the LAG+ immunotype were unlikely to respond to ICB with a 0% response rate compared to 49% for the LAG− and 27% for the PRO immunotypes (P=0.007). As in melanoma, these associations remained strong in the subset of patients who received anti-PD-1 monotherapy (n=67) (FIG. 7, panel C).

We next evaluated if any of the clinical features of the UC patient population correlated with the clusters defined by the flow cytometry analysis (Table 1). The age, gender, stage and prior treatment were all well balanced between the three clusters. In a multivariate analysis including immunotype, stage, LDH, and liver metastases, both the LAG+ and PRO immunotypes had significantly poorer outcomes (P<0.001, P=0.02, respectively) (FIG. 7, panel D). We also performed a multivariate analysis of immunotype and Bellmunt score in UC patients who progressed after platinum-based chemotherapy (n=86), and both the LAG+ and PRO immunotypes remained significant (P<0.001, P=0.05, respectively) (FIG. 8).

### Example 6. Immunotype is Independent of Previously Defined Immune Markers

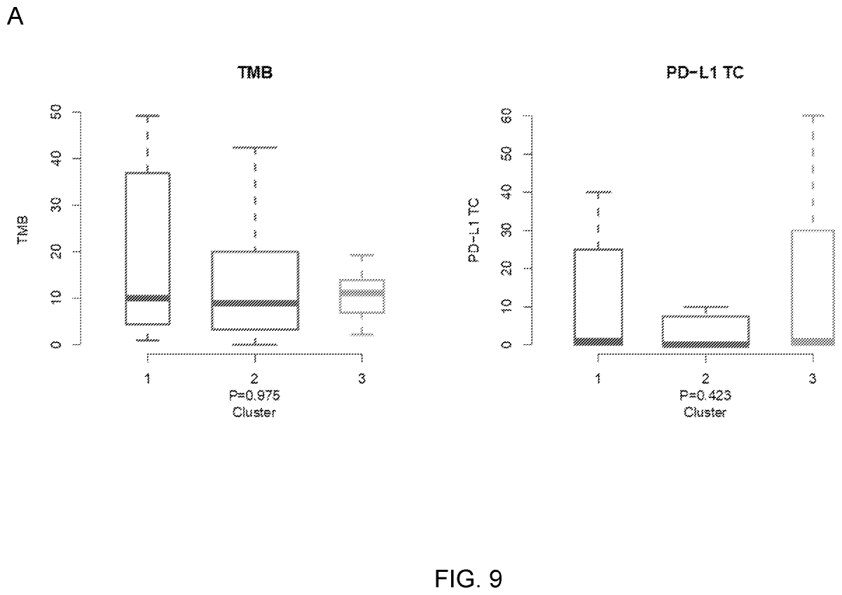

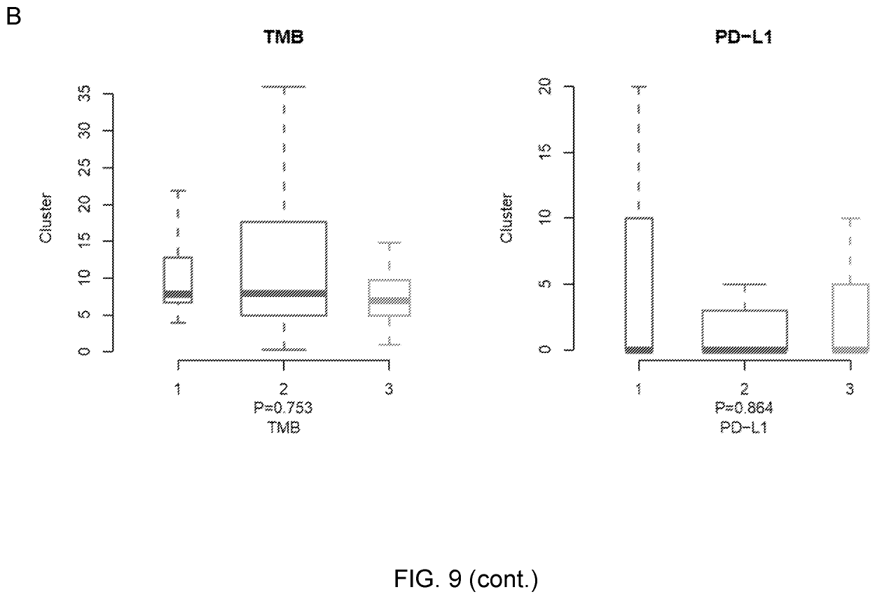

Tumor characteristics such as PD-L1 expression and TMB have been correlated with clinical outcomes in melanoma and UC patients treated with ICB. The distribution of these markers did not show any disproportionate allocation across the immunotypes (FIG. 9).

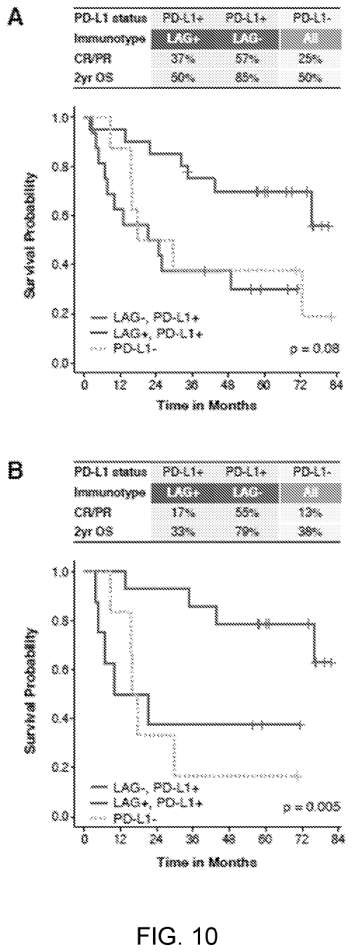

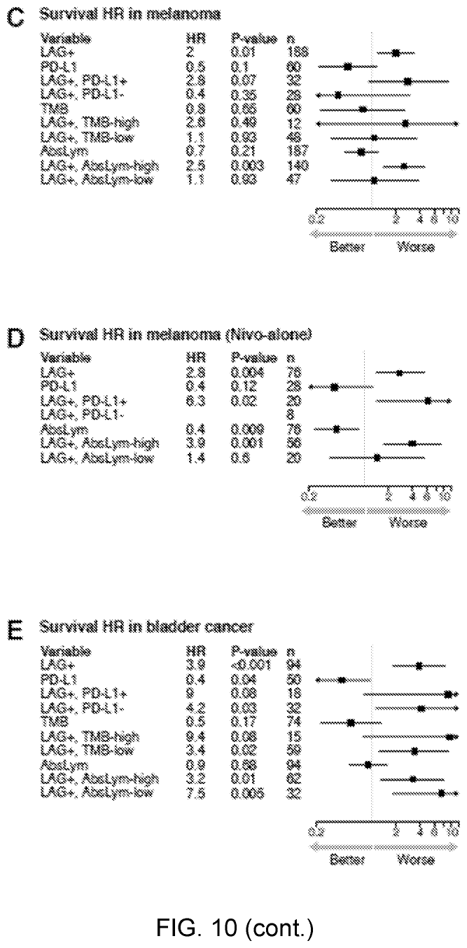

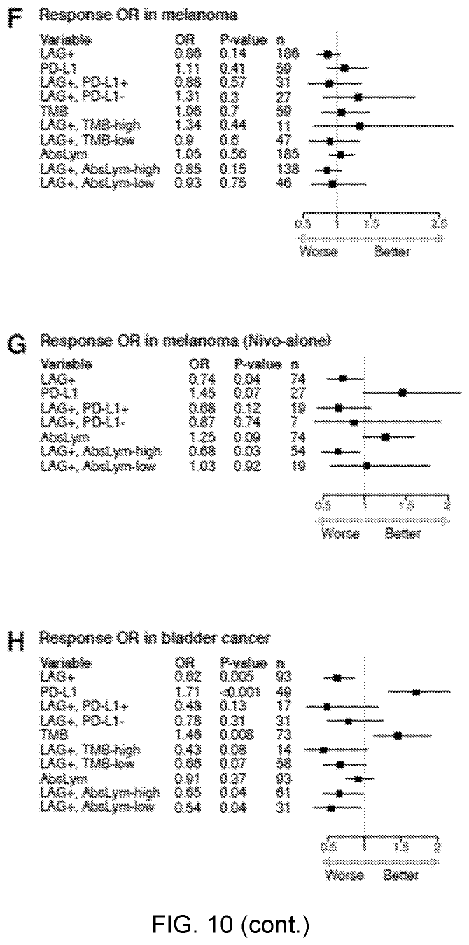

The LAG+ immunotype is associated with poor outcome regardless of PD-L1 or TMB status. In the melanoma cohort, PD-L1+ patients had significantly more favorable outcomes compared to the PD-L1-patients (median OS 75.8 v 23.0 months). However, amongst the PD-L1+ melanoma patients, those with the LAG+ immunotype had poorer survival outcomes and more closely resembled PD-L1-patients (FIG. 10, panels A and C). This pattern is even more striking in those who received anti-PD-1 monotherapy (FIG. 10, panels B and D).

While the PD-L1 and TMB data were more limited in the melanoma cohort (n=60) due to tissue availability from earlier trials, we could more confidently establish the relationship between TMB and LAG+ immunotype in the UC validation cohort. In particular, the favorable survival outcome conferred by either TMB-high or PD-L1+ status was abrogated amongst patients with the LAG+ immunotype (FIG. 10, panel E). Patients in the LAG+ immunotype also showed reduced response rate in PD-L1+ and TMB-high tumors (FIG. 10, panel H). Consistent with the literature,¹⁴ high absolute lymphocyte count (ALC) was a favorable factor for melanoma patients. However, in patients with high ALC, the LAG+ immunotype conferred poorer outcomes (FIG. 10, panels F and G).

### Example 7. Tables

TABLE 1
Clinical Characteristics of Patients in the Melanoma Discovery Cohort and Urothelial
Carcinoma Patient Cohort. According to Peripheral Blood Immunotype

| | Melanoma | | | | | | | | |
|---|---|---|---|---|---|---|---|---|---|
| | Overall | | LAG+ | | LAG− | | PRO | | |
| | N = | | N = | | N = | | N = | | P- |
| Characteristic | 136 | | 23¹ | | 89¹ | | 24¹ | | value² |
| Age | 62 | (22, 88) | 63 | (27, 88) | 61 | (22, 83) | 61 | (29, 80) | 0.8 |
| (median, | | | | | | | | | |
| range) | | | | | | | | | |
| Male | 82 | (60%) | 15 | (65%) | 54 | (61%) | 13 | (54%) | 0.7 |
| Stage | | | | | | | | | 0.11 |
| III | 6 | (4.4%) | 1 | (4.3%) | 5 | (5.6%) | 0 | (0%) | |
| IV M1a | 24 | (18%) | 2 | (8.7%) | 17 | (19%) | 5 | (21%) | |
| IV M1b | 23 | (17%) | 5 | (22%) | 18 | (20%) | 0 | (0%) | |
| IV M1c | 72 | (53%) | 13 | (57%) | 44 | (49%) | 15 | (62%) | |
| IV M1d | 11 | (8.1%) | 2 | (8.7%) | 5 | (5.6%) | 4 | (17%) | |
| IVA | | | | | | | | | |
| IVB³ | | | | | | | | | |
| Liver | 51 | (38%) | 10 | (43%) | 28 | (31%) | 13 | (54%) | 0.10 |
| metastases | | | | | | | | | |
| LDH | 205 | (172, 285) | 210 | (181, 288) | 195 | (168, 233) | 296 | (205, 508) | 0.002 |
| (median, | | | | | | | | | |
| IQR) | | | | | | | | | |
| Histology | | | | | | | | | 0.3 |
| Cutaneous/ | 106 | (78%) | 15 | (65%) | 71 | (80%) | 20 | (83%) | |
| Unknown | | | | | | | | | |
| Mucosal/ | 30 | (22%) | 8 | (35%) | 18 | (20%) | 4 | (17%) | |
| Acral/ | | | | | | | | | |
| Uveal | | | | | | | | | |
| Pure | | | | | | | | | |
| transitional | | | | | | | | | |
| cell | | | | | | | | | |
| Mixed | | | | | | | | | |
| histology | | | | | | | | | |
| Prior | 54 | (40%) | 8 | (35%) | 35 | (39%) | 11 | (46%) | 0.7 |
| Immunotherapy | | | | | | | | | |
| Prior | | | | | | | | | |
| Platinum | | | | | | | | | |
| Treatment | | | | | | | | | |
| Prior | | | | | | | | | |
| Intravesical | | | | | | | | | |
| BCG | | | | | | | | | |
| Bellmunt | | | | | | | | | |
| Prognostic | | | | | | | | | |
| Score | | | | | | | | | |
| 0 | | | | | | | | | |
| 1 | | | | | | | | | |
| 2+ | | | | | | | | | |
| Treatment | | | | | | | | | 0.6 |
| Anti-PD1 | 76 | (56%) | 15 | (65%) | 45 | (51%) | 16 | (67%) | |
| Anti-CTLA4 | 13 | (9.6%) | 2 | (8.7%) | 9 | (10%) | 2 | (8.3%) | |
| Combination | 47 | (35%) | 6 | (26%) | 35 | (39%) | 6 | (25%) | |
| | Urothelial Carcinoma | | | | | | | | |
| | Overall | | LAG+ | | LAG− | | PRO | | |
| | N = | | N = | | N = | | N = | | P- |
| Characteristic | 94 | | 9¹ | | 70¹ | | 15¹ | | value² |
| Age | 67 | (31, 83) | 69 | (50, 76) | 66 | (31, 83) | 66 | (51, 76) | 0.6 |
| (median, | | | | | | | | | |
| range) | | | | | | | | | |
| Male | 76 | (81%) | 8 | (89%) | 56 | (80%) | 12 | (80%) | >0.9 |
| Stage | | | | | | | | | >0.9 |
| III | 1 | (1.1%) | 0 | (0%) | 1 | (1.4%) | 0 | (0%) | |
| IV M1a | | | | | | | | | |
| IV M1b | | | | | | | | | |
| IV M1c | | | | | | | | | |
| IV M1d | | | | | | | | | |
| IVA | 29 | (31%) | 3 | (33%) | 22 | (31%) | 4 | (27%) | |
| IVB³ | 64 | (68%) | 6 | (67%) | 47 | (67%) | 11 | (73%) | |
| Liver | 26 | (28%) | 5 | (56%) | 15 | (21%) | 6 | (40%) | 0.045 |
| metastases | | | | | | | | | |
| LDH | 201 | (167, 237) | 180 | (153, 192) | 204 | (167, 236) | 211 | (184, 254) | 0.2 |
| (median, | | | | | | | | | |
| IQR) | | | | | | | | | |
| Histology | | | | | | | | | 0.3 |
| Cutaneous/ | | | | | | | | | |
| Unknown | | | | | | | | | |
| Mucosal/ | | | | | | | | | |
| Acral/ | | | | | | | | | |
| Uveal | | | | | | | | | |
| Pure | 72 | (77%) | 7 | (78%) | 56 | (80%) | 9 | (60%) | |
| transitional | | | | | | | | | |
| cell | | | | | | | | | |
| Mixed | 22 | (23%) | 2 | (22%) | 14 | (20%) | 6 | (40%) | |
| histology | | | | | | | | | |
| Prior | 0 | (0%) | 0 | (0%) | 0 | (0%) | 0 | (0%) | |
| Immunotherapy | | | | | | | | | |
| Prior | 86 | (91%) | 8 | (89%) | 64 | (91%) | 14 | (93%) | 0.8 |
| Platinum | | | | | | | | | |
| Treatment | | | | | | | | | |
| Prior | 33 | (35%) | 4 | (44%) | 25 | (36%) | 4 | (27%) | 0.6 |
| Intravesical | | | | | | | | | |
| BCG | | | | | | | | | |
| Bellmunt | | | | | | | | | 0.038 |
| Prognostic | | | | | | | | | |
| Score | | | | | | | | | |
| 0 | 33 | (35%) | 4 | (44%) | 28 | (40%) | 1 | (6.7%) | |
| 1 | 48 | (51%) | 4 | (44%) | 31 | (44%) | 13 | (87%) | |
| 2+ | 13 | (14%) | 1 | (11%) | 11 | (16%) | 1 | (6.7%) | |
| Treatment | | | | | | | | | 0.4 |
| Anti-PD1 | 67 | (71%) | 6 | (67%) | 48 | (69%) | 13 | (87%) | |
| Anti-CTLA4 | | | | | | | | | |
| Combination | 27 | (29%) | 3 | (33%) | 22 | (31%) | 2 | (13%) | |

¹Statistics presented: median (minimum, maximum); n (%); median (IQR)
²Statistical tests performed: Kruskal-Wallis test; Fisher's exact test
³Non-lymph node distant metastases

TABLE 2
Clinical Characteristics of Full Melanoma Dataset (n = 188), According to Immunotype

| Characteristic | Overall, N = 188 | LAG+, N = 31¹ | LAG−, N = 128¹ | PRO, N = 29¹ | P-value² |
|---|---|---|---|---|---|
| Age (median, range) | 62 (19, 88) | 65 (27, 88) | 61 (19. 83) | 64 (29, 80) | 0.3 |
| Male | 114 (61%) | 20 (65%) | 77 (60%) | 17 (59%) | 0.9 |
| Stage | | | | | |
| III | 16 (8.5%) | 5 (16%) | 11 (8.6%) | 0 (0%) | |
| IV M1a | 28 (15%) | 2 (6.5%) | 20 (16%) | 6 (21%) | |
| IV M1b | 30 (16%) | 5 (16%) | 25 (20%) | 0 (0%) | |
| IV M1c | 95 (51%) | 17 (55%) | 60 (47%) | 18 (62%) | |
| IV M1d | 19 (10%) | 2 (6.5%) | 12 (9.4%) | 5 (17%) | |
| Liver metastases | 67 (36%) | 13 (43%) | 40 (31%) | 14 (48%) | 0.15 |
| LDH (median, IQR) | 209 (175, 295) | 204 (175, 265) | 203 (171, 251) | 290 (205, 508) | 0.005 |
| Histology | | | | | 0.077 |
| Cutaneous/Unknown | 143 (76%) | 19 (61%) | 99 (77%) | 25 (86%) | |
| Mucosal/Acral/Uveal | 45 (24%) | 12 (39%) | 29 (23%) | 4 (14%) | |
| Prior Immunotherapy | 55 (29%) | 8 (26%) | 36 (28%) | 11 (38%) | 0.5 |
| Treatment | | | | | 0.2 |
| Anti-PD1 | 76 (40%) | 15 (48%) | 45 (35%) | 16 (55%) | |
| Anti-CTLA4 | 13 (6.9%) | 2 (6.5%) | 9 (7.0%) | 2 (6.9%) | |
| Combination | 99 (53%) | 14 (45%) | 74 (58%) | 11 (38%) | |

¹Statistics presented: median (minimum, maximum); n (8%); median (IQR)
²Statistical tests performed: Kruskal-Wallis test: Fisher's exact test

### Example 8. Materials and Methods

### Patients/Specimen Collection

Patients were consented for blood collection under an approved protocol in accordance with the Institutional Review Board of Memorial Sloan Kettering Cancer Center. Patients were included if they participated in one of the following clinical studies: NCT01024231, NCT01295827, NCT01621490, NCT01844505, NCT01927419, NCT01928394, NCT02083484, NCT02553642, NCT03122522. Patients were accrued between 2009 and 2019. Patients were excluded if they did not have banked samples (n=104) or if they did not have tumor response assessment or survival follow up (n=4). Response was determined by RECIST criteria. Best overall response was determined partial response (PR), complete response (CR), stable disease (SD), progression of disease (PD), except in cases where patients had clinical progression and no radiographic assessment (PD).

### Flow Cytometry

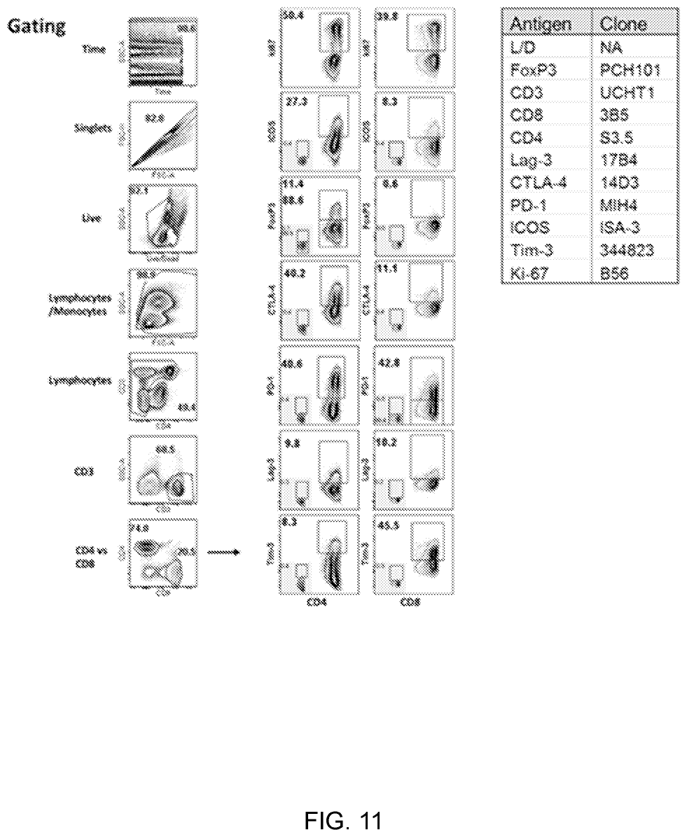

Cryopreserved peripheral blood mononuclear cells (PBMCs) were prepared and flow cytometry performed as previously described.¹⁰ Data were analyzed using Flow-jo software by an investigator blinded to clinical outcome (FIG. 11).

### Tumor Biospecimen Analysis

PD-L1 staining was performed according to institutional standard operating procedures using the Cell Signaling Technology antibody clone E1L3N. The proportion of PD-L1 positive tumor cells was determined by pathologists blinded to clinical outcome using a cutoff of 1%. TMB was estimated for tumors sequenced by the MSK-IMPACT platform as total mutation count per Mb sequenced; a cutoff of the highest 20% was used to define high TMB.¹¹

### Statistical Analysis

The survClust algorithm¹² was used to identify subgroups in the melanoma patient cohort based on the flow cytometry data. The glmnet algorithm was used to determine the 4-marker classifier for further validation of the immunotypes in the UC cohort. For clinical associations, Wilcoxon rank-sum test, Kruskal-Wallis test, or Fisher's exact test were used as appropriate. The Kaplan-Meier method was used for survival estimation and the long-rank test was used for comparisons. Cox proportional hazards model was used for association analysis with survival outcome for univariate and multivariate analysis, and for calculating the hazard ratio estimates along with 95% confidence intervals.

survClust Analysis

survClust is an outcome-weighted clustering algorithm for patient stratification building on ideas from supervised text classification.⁴¹ The algorithm learns a weighted distance matrix that down-weighs flow cytometry features that bear no relevance to the clinical outcome of interest. The immune cell phenotype in peripheral blood of cancer patients as measured by high dimensional flow cytometry is complex and influenced by a multitude of factors. Unsupervised learning does not necessarily lead to unique answers in highly complex data as many local optima may exist that pose special challenges in optimization. survClust overcomes the challenge by learning an outcome-weighted distance matrix from flow cytometry data incorporating a vector of hazard ratios estimated from Cox regression as weights. For a pair of two patient sample vector a and b, the weighted distance is calculated as follows:

d_{w}(a,b)=\sqrt{(a−b)^{T}W(a−b)}, (1)

where, a and b denote flow cytometry marker vectors of length p capturing the peripheral T cell phenotype profile in the corresponding PBMC sample, W is a p×p diagonal weight matrix with W=diag {w₁, . . . , wₚ}. The weights wⱼ (j=1, . . . , p) are obtained by fitting a univariate Cox proportional hazards model for each flow cytometry marker:

h(t|xₚ)=hₒ×exp(xⱼ^{T}*β), (2)

where t represents the survival time, xⱼ is the j^{th} column of matrix X of length N, h₀ is the baseline hazard function, β is the regression coefficient and exp(β) is the Hazard Ratio (HR). We consider the absolute value of HR on the logarithmic scale as the weight w. An HR=1 corresponds to the null that the feature is not associated with survival. This is reflected in a log(1)=0 weigh in the distance matrix. The weighted distance matrix is then projected onto a lower dimensional space via multidimensional scaling (MDS). Patient samples are then clustered into subgroups via the K-means algorithm in the MDS projected space. An implementation of survClust is publicly available at the github/com/arorarshi/survClust website. The number of clusters is determined through a 5-fold cross-validation (CV). Cluster identity is tracked by a centroid relabeling approach. The final class label for each sample is assigned through consensus of the prediction across all the CV rounds.

### Batch Correction

For each patient sample, we have information on the clinical trial ID, the time the sample was collected, and flow cytometry processing date. Overall, samples from the same clinical trial were collected in roughly the same time frame. We observed a moderate degree of “batch” effect by trial ID. Principal component analysis was used to visualize the pattern of the flow cytometry data across the batches. The ComBat algorithm was used to remove the “unwanted” variations due to batch based on an empirical Bayes approach.⁴⁰ The batch-adjusted data set was then used for subsequent analyses. No significant batch effect was observed in the UC data, but to be consistent with the adjustment, we applied the same ComBat analysis to remove any residual batch variation.

### Building a Classifier for Predicting the 3 Immunotypes

To derive a minimal set of markers for classifying future samples into the three T-cell immunotypes, we used the pairwise penalized logistic regression to select the most discriminative markers. We included LAG3, KI67, ICOS, TIM3, FOXP3, CTLA4, and PD1 on both CD4 and CD. For reproducibility, we excluded the three marker combinations, most of which will involve very small cell populations. FIG. 2 shows that LAG3CD8 was the most discriminant marker for distinguishing the LAG+vs LAG-immunotype. KI67, TIM3, and ICOS on CD8 were the most discriminant markers for further distinguishing the LAG+ from the PRO immunotype. Therefore, the combination of LAG3, KI67, TIM3, and ICOS on CD8 were used as the marker set for validation analysis. The glmnet R package was used for the marker selection analysis.⁴²

### Validation Analysis

A multinomial logistic regression was fitted using LAG3, K167, TIM3, and ICOS on CD8 and trained in the melanoma cohort. This model was then used to calculate predicted probability of each UC sample belonging to each of the three T-cell immunotypes. Each sample was then assigned to an immunotype based on maximum predicted probability. The nnet R package was used for this analysis.

### Software

All statistical analysis was performed in R version 3.6.3.43 Heatmaps were plotted using pheatmap and survival curves were drawn with the help of survminer R packages.^{44,45}

### REFERENCES

1. Andrews L P, Yano H, Vignali D A A. Inhibitory receptors and ligands beyond PD-1, P D-L1 and CTLA-4: breakthroughs or backups. Nat Immunol 2019; 20:1425-34.
2. Brignone C, Escudier B, Grygar C, Marcu M, Triebel F. A phase I pharmacokinetic and biological correlative study of IMP321, a novel MHC class II agonist, in patients with advanced renal cell carcinoma. Clin Cancer Res 2009; 15:6225-31.
3. Eastgate M A, Atkinson V, Khattak M A, et al. Pushing the accelerator and releasing the brake: A phase I dose escalation study evaluating a LAG-3 fusion protein (eftilagimod alpha), together with pembrolizumab in unresectable or metastatic melanoma. Journal of Clinical Oncology 36, no 15_suppl; 2018.
4. Ascierto P A, Melero I, Bhatia S, et al. Initial efficacy of anti-lymphocyte activation gene-3 (anti-LAG-3; BMS-986016) in combination with nivolumab (nivo) in pts with melanoma (MEL) previously treated with anti-PD-1/P D-L1 therapy. Journal of Clinical Oncology 35, no 15_suppl (May 20, 2017) 9520-9520 2017.
5. Harding J J, Patnaik A, Moreno V, et al. A phase Ia/Ib study of an anti-TIM-3 antibody (LY3321367) monotherapy or in combination with an anti-PD-L1 antibody (LY3300054): Interim safety, efficacy, and pharmacokinetic findings in advanced cancers. J Clin Oncol 37, 2019 (suppl 8; abstr 12); 2019.
6. Peguero J, Felip E, Bernard D, et al. Initial results from a Phase II study (TACTI-002) in non-small cell lung cancer, or head and neck cancer patients receiving eftilagimod alpha (LAG-3 fusion protein) and pembrolizumab. SITC; 2019.
7. Rodriguez-Abreu Dea. Primary analysis of a randomized, double-blind, phase II study of the anti-TIGIT antibody tiragolumab (tira) plus atezolizumab (atezo) versus placebo plus atezo as first-line (1L) treatment in patients with PD-L1-selected NSCLC (CITYSCAPE). ASCO; 2020.
8. Signorelli D, Giannatempo P, Grazia G, et al. Patients Selection for Immunotherapy in Solid Tumors: Overcome the Naive Vision of a Single Biomarker. Biomed Res Int 2019; 2019:9056417.
9. Davis A A, Patel V G. The role of PD-L1 expression as a predictive biomarker: an analysis of all US Food and Drug Administration (FDA) approvals of immune checkpoint inhibitors. J Immunother Cancer 2019; 7:278.
10. Huang A C, Postow M A, Orlowski R J, et al. T-cell invigoration to tumour burden ratio associated with anti-PD-1 response. Nature 2017; 545:60-5.
11. Samstein R M, Lee C H, Shoushtari A N, et al. Tumor mutational load predicts survival after immunotherapy across multiple cancer types. Nat Genet 2019; 51:202-6.
12. Arora A, Olshen A, Seshan V, Shen R. Pan-cancer identification of clinically relevant genomic subtypes using outcome-weighted integrative clustering. Biorxiv 2020.
13. Friedman J, Hastie T, Tibshirani R. Regularization Paths for Generalized Linear Models via Coordinate Descent. J Stat Softw 2010; 33:1-22.
14. Postow M A, Chasalow S D, Kuk D, et al. Absolute lymphocyte count as a prognostic biomarker for overall survival in patients with advanced melanoma treated with ipilimumab. Melanoma Res 2020; 30:71-5.
15. Snyder A, Makarov V, Merghoub T, et al. Genetic basis for clinical response to CTLA-4 blockade in melanoma. N Engl J Med 2014; 371:2189-99.
16. Rizvi N A, Hellmann M D, Snyder A, et al. Cancer immunology. Mutational landscape determines sensitivity to PD-1 blockade in non-small cell lung cancer. Science 2015; 348:124-8.
17. Taube J M, Klein A, Brahmer J R, et al. Association of PD-1, PD-1 ligands, and other features of the tumor immune microenvironment with response to anti-PD-1 therapy. Clin Cancer Res 2014; 20:5064-74.
18. Rosenberg J E, Hoffman-Censits J, Powles T, et al. Atezolizumab in patients with locally advanced and metastatic urothelial carcinoma who have progressed following treatment with platinum-based chemotherapy: a single-arm, multicentre, phase 2 trial. Lancet 2016; 387:1909-20.
19. Daud Al, Wolchok J D, Robert C, et al. Programmed Death-Ligand 1 Expression and Response to the Anti-Programmed Death 1 Antibody Pembrolizumab in Melanoma. J Clin Oncol 2016; 34:4102-9.
20. Herbst R S, Soria J C, Kowanetz M, et al. Predictive correlates of response to the anti-PD-L1 antibody MPDL3280A in cancer patients. Nature 2014; 515:563-7.
21. Garon E B, Rizvi N A, Hui R, et al. Pembrolizumab for the treatment of non-small-cell lung cancer. N Engl J Med 2015; 372:2018-28.
22. Vennapusa B, Baker B, Kowanetz M, et al. Development of a PD-L1 Complementary Diagnostic Immunohistochemistry Assay (SP142) for Atezolizumab. Appl Immunohistochem Mol Morphol 2019; 27:92-100.
23. Schmid P, Rugo H S, Adams S, et al. Atezolizumab plus nab-paclitaxel as first-line treatment for unresectable, locally advanced or metastatic triple-negative breast cancer (IMpassion130): updated efficacy results from a randomised, double-blind, placebo-controlled, phase 3 trial. Lancet Oncol 2020; 21:44-59.
24. Balar A V, Castellano D, O'Donnell P H, et al. First-line pembrolizumab in cisplatin-ineligible patients with locally advanced and unresectable or metastatic urothelial cancer (KEYNOTE-052): a multicentre, single-arm, phase 2 study. Lancet Oncol 2017; 18:1483-92.
25. Balar A V, Galsky M D, Rosenberg J E, et al. Atezolizumab as first-line treatment in cisplatin-ineligible patients with locally advanced and metastatic urothelial carcinoma: a single-arm, multicentre, phase 2 trial. Lancet 2017; 389:67-76.
26. Grosso J F, Goldberg M V, Getnet D, et al. Functionally distinct LAG-3 and PD-1 subsets on activated and chronically stimulated CD8 T cells. J Immunol 2009; 182:6659-69.
27. Woo S R, Turnis M E, Goldberg M V, et al. Immune inhibitory molecules LAG-3 and PD-1 synergistically regulate T-cell function to promote tumoral immune escape. Cancer Res 2012; 72:917-27.
28. Kraman M, Faroudi M, Allen N L, et al. FS118, a Bispecific Antibody Targeting LAG-3 and PD-L1, Enhances T-Cell Activation Resulting in Potent Antitumor Activity. Clin Cancer Res 2020.
29. Grosso J F, Kelleher C C, Harris T J, et al. LAG-3 regulates CD8+T cell accumulation and effector function in murine self- and tumor-tolerance systems. J Clin Invest 2007; 117:3383-92.
30. Krieg C, Nowicka M, Guglietta S, et al. High-dimensional single-cell analysis predicts response to anti-PD-1 immunotherapy. Nat Med 2018; 24:144-53.
31. Subrahmanyam P B, Dong Z, Gusenleitner D, et al. Distinct predictive biomarker candidates for response to anti-CTLA-4 and anti-PD-1 immunotherapy in melanoma patients. J Immunother Cancer 2018; 6:18.
32. Bochem J, Zelba H, Amaral T, et al. Peripheral PD-1+CD56+T-cell frequencies correlate with outcome in stage IV melanoma under PD-1 blockade. PLoS One 2019; 14:e0221301.
33. Wistuba-Hamprecht K, Martens A, Heubach F, et al. Peripheral CD8 effector-memory type 1 T-cells correlate with outcome in ipilimumab-treated stage IV melanoma patients. Eur J Cancer 2017; 73:61-70.
34. Zappasodi R, Budhu S, Hellmann M D, et al. Non-conventional Inhibitory CD4(+)Foxp3(−)PD-1(hi) T Cells as a Biomarker of Immune Checkpoint Blockade Activity. Cancer Cell 2018; 34:691.
35. Spitzer M H, Carmi Y, Reticker-Flynn N E, et al. Systemic Immunity Is Required for Effective Cancer Immunotherapy. Cell 2017; 168:487-502 e15.
36. Martens A, Wistuba-Hamprecht K, Geukes Foppen M, et al. Baseline Peripheral Blood Biomarkers Associated with Clinical Outcome of Advanced Melanoma Patients Treated with Ipilimumab. Clin Cancer Res 2016; 22:2908-18.
37. Kagamu H, Kitano S, Yamaguchi O, et al. CD4(+) T-cell Immunity in the Peripheral Blood Correlates with Response to Anti-PD-1 Therapy. Cancer Immunol Res 2020; 8:334-44.
38. Fairfax B P, Taylor C A, Watson R A, et al. Peripheral CD8(+) T cell characteristics associated with durable responses to immune checkpoint blockade in patients with metastatic melanoma. Nat Med 2020; 26:193-9.
39. Ohmura H, Yamaguchi K, Hanamura F, et al. OX40 and LAG3 are associated with better prognosis in advanced gastric cancer patients treated with anti-programmed death-1 antibody. Br J Cancer 2020; 122:1507-17.
40. Johnson W E, Li C, Rabinovic A. Adjusting batch effects in microarray expression data using empirical Bayes methods. Biostatistics. 2007 Jan. 1; 8(1):118-27.
41. Arora A, Olshen A B, Seshan V E, and Shen R. Pan-cancer identification of clinically relevant genomic subtypes using outcome-weighted integrative clustering. Biorxiv 2020 May 11:084798.
42. Friedman J, Hastie T, Tibshirani R. Regularization paths for generalized linear models via coordinate descent. Journal of statistical software. 2010; 33(1):1.
43. R Core Team (2020). R: A language and environment for statistical computing. R Foundation for Statistical Computing, Vienna, Austria. See website at R-project.org.
44. Raivo Kolde (2019). pheatmap: Pretty Heatmaps. R package version 1.0.12. See website at CRAN.R-project.org/package=pheatmap.
45. Alboukadel K, Marcin K, Przemyslaw B. (2019). survminer: Drawing Survival Curves using ‘ggplot2’. R package version 0.4.6. See website at CRAN.R-project.org/package=survminer.
46. Ollivier L, Padhani A R, Leclere, J. International criteria for measurement of tumor response. Cancer Imaging 2001; 2(1):31-32.
47. Andrews L P, et al. Immunol. Rev. 2017; 276(1): 80-96.
48. Puhr H C, et al. ESMO Open 2019; 4: e000482.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A method of detecting a LAG+, LAG−, or PRO immunotype in a cancer patient, the method comprising:
a. conducting flow cytometry on a blood sample from the patient to determine normalized frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in the blood sample; and
b. implementing a classifier algorithm on a programmed computer, wherein the classifier algorithm uses a multinomial logistic regression to predict probabilities of the patient belonging to an immunotype of LAG+, LAG−, or PRO, by comparing the normalized frequencies determined in (a) with frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, from a training set of immunotype-labeled frequencies, wherein the training set is from a population of control cancer patients treated with immune checkpoint blockade (ICB) therapy;
wherein the classifier algorithm assigns an immunotype of LAG+, LAG−, or PRO to the patient, based on the immunotype that has the highest predicted probability.

2. A method of treating cancer in a patient, the method comprising:
a. prior to anti-cancer therapy, detecting an immunotype of LAG+, LAG−, or PRO in the patient by the method according to claim 1; and
b. (i) if the patient has a LAG− immunotype, administering a PD-1 inhibitor monotherapy to the patient; or
(ii) if the patient has a LAG+ or PRO immunotype, administering anti-cancer therapy that is not PD-1 inhibitor monotherapy.

3. The method of claim 2, comprising treating the patient having a LAG+ or PRO immunotype with immune checkpoint blockade (ICB) combination therapy or ICB monotherapy, wherein the ICB monotherapy is not PD-1 inhibitor monotherapy.

4. A method for predicting a response to PD-1 inhibitor monotherapy in a cancer patient, the method comprising classifying the cancer patient as having an immunotype selected from LAG+, LAG−, and PRO, wherein the classifying comprises:
a. determining normalized frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in a blood sample from the patient; and
b. implementing a classifier algorithm that uses a multinomial logistic regression to predict probabilities of the patient belonging to an immunotype of LAG+, LAG−, or PRO, by comparing the normalized frequencies determined in (a) with frequencies of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, from a training set of immunotype-labeled frequencies, wherein the training set is from a population of control cancer patients treated with immune checkpoint blockade (ICB) therapy;
wherein a cancer patient having a LAG− immunotype is predicted to be susceptible to PD-1 inhibitor monotherapy and wherein a cancer patient having a LAG3+ or PRO immunotype is predicted to be less susceptible to PD-1 inhibitor monotherapy, thereby predicting a response to PD-1 inhibitor monotherapy.

5. The method of claim 4, further comprising treating the cancer patient having a LAG− immunotype with PD-1 inhibitor monotherapy.

6. The method of claim 4, further comprising treating the cancer patient having a LAG+ or PRO immunotype with anti-cancer therapy, wherein the anti-cancer therapy is not PD-1 inhibitor monotherapy.

7. A method of treating cancer in a patient, the method comprising administering to a patient having a LAG− immunotype PD-1 inhibitor monotherapy.

8. A method of treating cancer in a patient, the method comprising administering to a patient having a LAG+ or PRO immunotype anti-cancer therapy, wherein the anti-cancer therapy is not PD-1 inhibitor monotherapy.

9. The method of any one of claims 3, 6, or 8, comprising treating the cancer patient having a LAG+ immunotype with ICB therapy comprising a LAG-3 inhibitor.

10. The method of claim 9, comprising treating the cancer patient with combination ICB therapy comprising a PD-1 inhibitor.

11. Use of the frequency of (i) LAG-3+CD8+T-cells, (ii) Ki67+CD8+T-cells, (iii) Tim-3+CD8+T-cells, and (iv) ICOS+CD8+T-cells, as a percentage of total CD8+T-cells, in a blood sample from a patient as a biomarker for success of ICB therapy in a cancer patient.

12. The use of claim 11, wherein the ICB therapy comprises a PD-1 inhibitor.

13. The use of claim 11 or claim 12, wherein the ICB therapy comprises a LAG-3 inhibitor.

14. The method or use of any one of claims 2, 3, 6, or 8 to 13, wherein the anti-cancer therapy or ICB therapy comprises a CTLA-4 inhibitor.

15. The method or use of any one of claims 2, 4, 5, 7, 10, or 12, wherein the PD-1 inhibitor is selected from the group consisting of nivolumab, pembrolizumab, pidilizumab, and REGN2810.

16. The method or use of any one of claims 2, 4, 5, 7, 10, or 12, wherein the PD-1 inhibitor is selected from the group consisting of atezolizumab, avelumab, durvalumab, and BMS-936559.

17. The method or use of claim 14, wherein the CTLA-4 inhibitor is selected from the group consisting of ipilimumab and tremelimumab.

18. The method or use of claim 9 or claim 13, wherein the LAG-3 inhibitor is selected from the group consisting of EOC202, FS118, GSK2831781, INCAGNO2385, IMP321, LAG525, MGD013, MK-4280, REGN3767, relatilimab (BMS986016), Sym-022, and TSR-033.

19. The method or use of any one of claims 3 to 18, wherein T cell frequency is measured using flow cytometry.

20. The method or use of claim 19, wherein the flow cytometry is fluorescence-activated cell sorting (FACS).

21. The method or use of any one of claims 1 to 20, wherein the cancer is melanoma.

22. The method or use of any one of claims 1 to 20, wherein the cancer is urothelial carcinoma.
